(19) United States
(12) Patent Application Publication
(10) Pub. No.: US 2003/0086887 A1
(43) Pub. Date: May 8, 2003

(54) Film-forming cosmetic composition

(76) Inventors: Valerie De La Poterie, Le Chatelet en Brie (FR); Frederic Auguste, Chevilly-Larue (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/176,642

(22) Filed: Jun. 24, 2002

(30) Foreign Application Priority Data
Jun. 22, 2001 (FR) 01 08286

(51) Int. Cl. A61K 7/06; A61K 7/11

(52) U.S. Cl. 424/70.11

(57) ABSTRACT

Use of a hydrophobic film-forming polymer and of a nonionic organic compound with a melting point ranging from 20° C. to 70° C. and an IOB (Inorganic/Organic Balance) value of less than or equal to 0.3, in a composition comprising a physiologically acceptable medium, to obtain a film applied to a keratin material resistant to cold water and/or which may be removed with warm water. A composition comprising, in a physiologically acceptable medium, at least one hydrophobic polymer, and at least one nonionic organic compound with a melting point ranging from 20° C. to 70° C. and an IOB (Inorganic/Organic Balance) value of less than or equal to 0.3. Use of the composition for making up and caring for a keratin material.

The present invention relates to the use of a hydrophobic film-forming polymer and a special organic compound to obtain a film which can be removed with warm water.

The invention also relates to a make-up or cosmetic care composition for keratin materials such as the skin, the eyelashes, the eyebrows, the hair and the nails, such as those of human beings, comprising a hydrophobic film-forming polymer and a special organic compound, and also to a cosmetic care or make-up process for keratin materials.

The composition may be in the form of a mascara, an eyeliner, a product for the lips, a blusher, an eyeshadow, a foundation, a make-up product for the body, a concealer product, a product for the nails, an antisun composition, a skin colouring composition or a skincare product. The invention can relate to a mascara, for instance.

Mascara compositions in the form of a wax-in-water emulsion comprising surfactants are known from document WO-A-95/15741, the disclosure of which is incorporated herein by reference. However, the make-up film obtained with these compositions may not show good water resistance and when the film comes into contact with water, for example when bathing or taking a shower, it may partially disintegrate by being worn away or by spreading around the eyes. The wearing away of the film gives rise to a substantial reduction in the intensity of the colour of the make-up, thus obliging the consumer to freshen the application of the mascara. As regards the spreading of the film, this forms a very unsightly aureole around the area to which make-up has been applied. Tears and perspiration can also cause these same drawbacks.

To promote the water resistance of make-up, it is known practice from document U.S. Pat. No. 4,423,031, the disclosure of which is incorporated herein by reference, to use acrylic polymers in aqueous dispersion. However, the mascara may be difficult to remove and can require the use of special make-up removers based on oils or on organic solvents. Now, these make-up removers may be irritant to the eyes, they may cause stinging or they may leave a veil over the eyes, or alternatively they may leave an uncomfortable greasy residual film on the skin around the eyes (eyelids).

To avoid the use of these special make-up removers, it is possible to use soap and water, as disclosed in document WO-A-96/33690, the disclosure of which is incorporated herein by reference, by proposing a mascara comprising a water-insoluble polymer and a water-soluble film-forming polymer. However, the use of soap may cause eye discomfort due to stinging or to the deposition of a veil over the eyes. Soap can also dissolve the film of make-up, which then spreads around the eyes, forms unsightly aureoles, and stains the skin.

The use of warm water, defined herein as water with a temperature of at least 35° C. (temperature measured at atmospheric pressure), such as temperatures ranging from about 35° C. to 50° C., makes it possible to avoid the drawbacks of the make-up removers known hitherto, but the cold-water-resistant mascara compositions described previously cannot be removed with warm water.

The aim of the present invention is thus to propose a cosmetic composition which forms a film which may be removed with warm water while at the same time having good cold-water resistance.

The inventors have discovered that such a film may be obtained using a film-forming polymer capable of forming a hydrophobic film and a particular fatty substance. After applying the film to a keratin material, such as the eyelashes, the film of make-up obtained shows good resistance to cold water, defined herein as water with a temperature below or equal to 30° C., for example when bathing, and/or to tears and/or to perspiration. The film of make-up is easily removed with warm water, such as by rubbing with cotton wool or a gauze: the film of make-up can detach easily from the eyelashes and can be removed from the eyelashes without fragmenting (in the form of a sheath) or in the form of fragments or pieces. The film of make-up thus removed does not spread on the skin which avoids the formation of aureoles around the eye; the skin is not stained when removing the make-up and remains clean. The film of make-up is removed very simply with warm water and with warm water containing no detergent such as soaps, for example. For the make-up removal, the warm water used may be tap water, demineralized water or mineral water brought to a temperature of at least 35° C., such as temperatures ranging from about 35° C. to 50° C.

A subject of the invention is the use of a hydrophobic film-forming polymer and of a nonionic organic compound with an IOB (Inorganic/Organic Balance) value of less than or equal to 0.3, to obtain a film applied to keratin materials which may be removed with warm water.

A subject of the invention is also a composition comprising, in a physiologically acceptable medium, at least one hydrophobic film-forming polymer and at least one nonionic organic compound with an IOB (Inorganic/Organic Balance) value of less than or equal to 0.3.

A subject of the invention is also the use of a composition as defined above to obtain a film applied to a keratin material which film is resistant to cold water and/or may be removed with warm water.

A subject of the invention is also a cosmetic care or make-up process for a keratin material, comprising the application of a composition as defined above to a keratin material.

A subject of the invention is also a cosmetic process for removing a film of make-up from a keratin material made up with a composition as defined above, comprising at least one step of rinsing the said made-up keratin material with warm water, i.e. water maintained at a temperature of at least 35° C.

The expression “physiologically acceptable medium” should be understood as meaning a medium which is compatible with keratin materials, such as a cosmetic medium.

For example, the composition according to the invention contains little emulsifier (surfactant), or is even free of emulsifier, for example, an amount of less than 0.5% by weight relative to the total weight of the composition. The composition thus has good resistance to cold water.

The term “emulsifier” means any amphiphilic compound chosen from nonionic amphiphilic compounds with an HLB (hydrophilic-lipophilic balance) of greater than or equal to 10 and ionic amphiphilic compounds whose hydrophilic portion comprises a counterion with a molar mass of greater than or equal to 50 g/mol.

The make-up removal of the film with warm water is obtained using an organic compound with a melting point ranging from 20° C. to 70° C. and a particular IOB (Inorganic/Organic Balance) value. This particular organic compound makes the polymer film more water-sensitive: the make-up film is made brittle on contact with warm water and by rubbing it, for example, with the fingers or with a cloth or cotton wool, and the film disintegrates readily or detaches from its support.

The organic compound has a melting point ranging from 20° C. to 70° C. This melting point may be measured by any known method, such as by means of differential scanning calorimetry (DSC).

The said organic compound has an IOB (Inorganic/Organic Balance) value of less than or equal to 0.3, such as a range of from 0.05 to 0.3, and further such as from 0.1 to 0.2.

The expression “organic compound with an IOB value of less than or equal to 0.3” means a carbon-based organic compound which by itself has the said IOB value or a mixture of carbon-based organic compounds, the said mixture having the said IOB value. In the said mixture, each organic compound must have a melting point ranging from 20° C. to 70° C. as described previously. For example, the said organic compound with an IOB value of less than or equal to 0.3 is a single organic compound having the said IOB value.

The IOB parameter is known to those skilled in the art from a certain number of publications, the disclosures of which are incorporated herein by reference, for instance:

(1) “Prediction of organic compounds by a conceptional diagram”, A. FUJITA Pharm. Bull 2, 163-173 (1954)

(2) “Organic Analysis” Fujita (1930), published by Kaniya Shoten,

(3) “Prediction of Organic Compounds and Organic Conceptional Diagram” A. Fujita (Kagaku-no-Ryoiki 11-10)” (1957), pp. 719-725,

(4) “Systematic Organic Qualitative Analysis (Book of Purified Substances)” Fujita and Akatsuka (1970), p. 487, published by Kazama Shoten,

(5) “Organic Conceptional Diagram, Its Fundamentals and applications”, Koda (1984), p. 227, published by Sankyo Shuppan,

(6) “Design of Emulsion Formulations by use of Organic Conceptional Diagram” (1985), p. 98, Yaguchi, published by Nippon Emulsion K.K.,

(7) R. H. Ewell, J. M. Harrison, L. Berg.: Ind. Eng. Chem. 36, 871 (1944),

(8) EP-A-985404

The IOB of a compound corresponds to the ratio of the inorganic value of the compound to the organic value of the compound:

IOB=inorganic value/organic value.

To calculate the organic value of a compound, the methylene group is considered as a unit and is evaluated by the number of carbon atoms. A carbon atom or a —CH3, —CH2— or ═CH— group is counted for a value of 20 (value without units). The hydrogen atoms are not taken into account. The presence of a ring, branching or an ethylenic or acetylenic unsaturation in the said organic compound is taken into account in calculating the organic value of the compound according to the corresponding organic value known in the literature, such as on page 167 of publication (1) mentioned above.

To calculate the inorganic value of a compound, the hydroxyl group is taken as the standard group, for which an inorganic value of 100 is attributed. This arbitrary value of 100 is correlated to the distance between the boiling point curve for the alkane series as a function of the number of carbon atoms in the said alkane and the boiling point curve for the linear saturated primary monoalcohols analogous to the alkanes.

The inorganic value (noted as Ix) of a substituent X (that is to say of any atom other than carbon or hydrogen, and of any group of atoms other than the groups of atoms formed exclusively of carbon and/or of hydrogen) is determined by means of graphs. This value Ix is calculated by determining, firstly, the boiling point (b.p.) of a linear alkane and the boiling point (b.p.x) of the homologue of the said linear alkane substituted with the substituent X, and then by calculating the difference ΔTx=b.p.x−b.p., and secondly by determining the boiling point (b.p.OH) of the homologue substituted with a primary alcohol group, and then by calculating the difference ΔTOH=b.p.OH−b.p. The value Ix is equal to the ratio of the difference ΔTx over ΔTOH, the said ratio all being multiplied by the inorganic value of the hydroxyl group, equal to 100.

$$Ix = \frac{Δ {T}_{X}}{Δ {T}_{OH}} \times 100$$

The inorganic values of many substituents are described in the literature, such as in those references cited previously. The inorganic value of a compound is calculated by adding the inorganic value of the (or all of the) substituent(s) present in the said compound.

Certain substituents, such as —Cl or —F, have both an organic value and an inorganic value, as indicated in reference (1) cited above on page 167, for example.

The IOB of a mixture of organic compounds is equal to the ratio of the sum of the inorganic values of the said organic compounds in the mixture to the sum of the organic values of the said organic compounds in the mixture.

For example, the said organic compound has an organic value ranging from 330 to 700, such as from 365 to 700, and further such as from 365 to 500.

For example, the said organic compound can have an inorganic value of less than or equal to 110, such as ranging from 0 to 110, and further such as ranging from 50 to 80.

The said organic compound may be chosen from esters, such as esters comprising from 18 to 36 carbon atoms, and monoalcohols comprising from 18 to 36 carbon atoms.

Thus, a subject of the invention is also the use of a hydrophobic film-forming polymer and of an organic compound chosen from esters comprising from 18 to 36 carbon atoms and monoalcohols comprising from 18 to 36 carbon atoms, to obtain a film applied to keratin materials that may be removed with warm water.

A subject of the invention is also a composition comprising, in a physiologically acceptable medium, at least one hydrophobic film-forming polymer and at least one organic compound chosen from esters comprising from 18 to 36 carbon atoms and monoalcohols comprising from 18 to 36 carbon atoms.

As organic compounds that may be used according to the invention, mention may be made of butyl stearate (IOB=0.136; organic value=440; inorganic value=60), cetyl palmitate (IOB=0.094; organic value=640; inorganic value=60), methyl palmitate (IOB=0.176; organic value=340; inorganic value=60), methyl stearate (IOB=0.158; organic value=380; inorganic value=60), stearyl alcohol (IOB=0.278; organic value=360; inorganic value=100).

For example, the organic compound may have a molecular weight ranging from 180 to 1,000.

The organic compound may be present in the composition in a content ranging from 0.1% to 20% by weight relative to the total weight of the composition, such as ranging from 0.5% to 15% by weight, and further such as ranging from 1% to 10% by weight.

According to the invention, the composition may also comprise a film-forming polymer capable of forming a hydrophobic film. In the present application, the term “film-forming polymer” means a polymer which is capable, by itself or in the presence of an auxiliary film-forming agent, of forming a continuous film which adheres to a support, such as to a keratin material.

The term “film-forming polymer capable of forming a hydrophobic film” means a polymer whose film has a solubility in water at 25° C. of less than 1% by weight.

The film-forming polymer may be chosen from synthetic polymers, such as free-radical polymers or polycondensates, and polymers of natural origin, and mixtures thereof.

The term “free-radical film-forming polymer” means a polymer obtained by polymerization of monomers containing unsaturation, such as ethylenic unsaturation (unlike polycondensates). The film-forming polymers of free-radical type may be vinyl polymers or copolymers, for example, such as acrylic polymers.

The vinyl film-forming polymers may result from the polymerization of monomers with ethylenic unsaturation comprising at least one acidic group and/or esters of these acid monomers and/or amides of these acid monomers.

As monomers bearing an acidic group, it is possible to use α,β-ethylenic unsaturated carboxylic acids such as acrylic acid, methacrylic acid, crotonic acid, maleic acid, or itaconic acid. (Meth)acrylic acid and crotonic acid can be used, for instance.

The esters of acid monomers can be chosen from (meth)acrylic acid esters (also referred to as (meth)acrylates), alkyl (meth)acrylates such as of a C1-C30 alkyl, and such as of a C1-C20 alkyl, which may be linear, branched or cyclic, aryl (meth)acrylates such as of a C6-C10 aryl, and hydroxyalkyl (meth)acrylates such as of a C2-C6 hydroxyalkyl.

Among the alkyl (meth)acrylates which may be mentioned are methyl methacrylate, ethyl methacrylate, butyl methacrylate, isobutyl methacrylate, 2-ethylhexyl methacrylate, lauryl methacrylate, and cyclohexyl (meth)acrylate.

Among the hydroxyalkyl (meth)acrylates which may be mentioned are hydroxyethyl acrylate, 2-hydroxypropyl acrylate, hydroxyethyl methacrylate, and 2-hydroxypropyl methacrylate.

Among the aryl (meth)acrylates which may be mentioned are benzyl acrylate and phenyl acrylate. The (meth)acrylic acid esters that may be used are the alkyl (meth)acrylates.

According to the present invention, the alkyl group of the esters may be either fluorinated or perfluorinated, that is to say that some or all of the hydrogen atoms of the alkyl group are replaced with fluorine atoms.

Examples of amides of the acid monomers which may be mentioned are (meth)acrylamides, such as N-alkyl(meth)acrylamides of a C1-C20 alkyl, for instance. Among the N-alkyl(meth)acrylamides which may be mentioned are N-ethylacrylamide, N-t-butylacrylamide, N-t-octylacrylamide, and N-undecylacrylamide.

The vinyl film-forming polymers may also result from the homopolymerization or copolymerization of at least one monomer chosen from vinyl esters, olefins (including fluoroolefins), vinyl ethers, and styrene monomers. For example, these monomers may be polymerized with acid monomers and/or esters thereof and/or amides thereof, such as those mentioned above.

Examples of vinyl esters which may be mentioned are vinyl acetate, vinyl neodecanoate, vinyl pivalate, vinyl benzoate, and vinyl t-butylbenzoate.

Among the olefins which may be mentioned are ethylene, propylene, butene, isobutene, octene, octadecene, polyfluorinated olefins such as tetrafluoroethylene, vinylidene fluoride, hexafluoropropene, or chlorotrifluoroethylene.

Styrene monomers which may be mentioned are styrene and α-methylstyrene.

As polycondensates which may be used as film-forming polymer, polyurethanes may be used, such as anionic, cationic, nonionic, or amphoteric polyurethanes, polyurethane-acrylics, polyurethane-polyvinylpyrrolidones, polyester-polyurethanes, polyether-polyurethanes, polyureas, polyurea/polyurethanes, and mixtures thereof.

The film-forming polyurethane may be, for example, an aliphatic, cyclic, or aromatic polyurethane, polyurea/urethane or polyurea copolymer, comprising, alone or as a mixture:

at least one sequence of aliphatic and/or cyclic and/or aromatic polyester origin, and/or

at least one branched or unbranched silicone sequence, for example polydimethylsiloxane or polymethylphenylsiloxane, and/or

at least one sequence comprising fluorinated groups.

Among the film-forming polycondensates which may also be mentioned are polyesters, polyesteramides, fatty-chain polyesters, polyamides, epoxyester resins, resins resulting from the condensation of formaldehyde with an arylsulphonamide, and arylsulphonamide-epoxy resins.

The polyesters may be obtained, in a known manner, by polycondensation of dicarboxylic acids with polyols, such as diols.

The dicarboxylic acid may be aliphatic, alicyclic, or aromatic. Examples of such acids which may be mentioned are: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, diglycolic acid, thiodipropionic acid, 2,5-naphthalenedicarboxylic acid, or 2,6-naphthalenedicarboxylic acid. These dicarboxylic acid monomers may be used alone or as a combination of at least two dicarboxylic acid monomers. Among these monomers, phthalic acid, isophthalic acid, and terephthalic acid can be chosen.

The diol may be chosen from aliphatic, alicyclic, and aromatic diols. The diol used can be chosen from: ethylene glycol, diethylene glycol, triethylene glycol, 1,3-propanediol, cyclohexanedimethanol, and 4-butanediol. Other polyols which may be used are glycerol, pentaerythritol, sorbitol, and trimethylolpropane.

The polyesteramides may be obtained in a manner similar to that of the polyesters, by polycondensation of diacids with diamines or with amino alcohols. Diamines which may be used include ethylenediamine, hexamethylenediamine, meta-phenylenediamine, and para-phenylenediamine. An amino alcohol which may be used is monoethanolamine.

The polyester may also comprise at least one monomer bearing at least one —SO3M group, with M representing a hydrogen atom, an ammonium ion NH4+, or a metal ion such as, for example, an Na+, Li+, K+, Mg2+, Ca2+, Cu2+, Fe2+or Fe3+ ion. A difunctional aromatic monomer comprising such a —SO3M group may also be used.

The aromatic nucleus of the difunctional aromatic monomer also bearing a —SO3M group as described above may be chosen, for example, from benzene, naphthalene, anthracene, biphenyl, oxybiphenyl, sulphonylbiphenyl, and methylenebiphenyl nuclei. Examples of difunctional aromatic monomers also bearing a —SO3M group which may be mentioned include: sulphoisophthalic acid, sulphoterephthalic acid, sulphophthalic acid, and 4-sulphonaphthalene-2,7-dicarboxylic acid.

In the compositions which are the subject of the invention, copolymers based on isophthalate/sulphoisophthalate may be used, such as copolymers obtained by condensation of di-ethylene glycol, cyclohexanedimethanol, and isophthalic acid or sulphoisophthalic acid. Such polymers are sold, for example, under the brand name Eastman AQ by the company Eastman Chemical Products.

The synthetic hydrophobic polymer may also be a silicone polymer, for example polyorganopolysiloxane.

The polymers of natural origin, which are optionally modified, may be chosen from shellac resin, sandarac gum, dammar resins, elemi gums, copal resins, cellulose polymers such as nitrocellulose, cellulose acetate, cellulose acetobutyrate, cellulose acetopropionate or ethylcellulose, and mixtures thereof.

According to an embodiment of the invention, the first film-forming polymer may be present in the form of solid particles dispersed in an aqueous medium. The expression “polymer in the form of particles in aqueous dispersion”, which is generally known as a latex or pseudolatex, means a phase containing water and optionally a water-soluble compound, in which is directly dispersed the polymer in the form of particles.

The size of the polymer particles in aqueous dispersion may be from 10 nm to 500 nm, such as from 20 nm to 300 nm. One skilled in the art can use known methods to determine these sizes.

The aqueous medium may comprise water or may also comprise a mixture of water and of water-miscible solvent, for instance lower monoalcohols comprising from 1 to 5 carbon atoms, glycols comprising from 2 to 8 carbon atoms, C3-C4 ketones or C2-C4 aldehydes. In practice, the aqueous medium represents from 5% to 94.9% by weight relative to the total weight of the composition.

Film-forming polymers in aqueous dispersion which may be used include the acrylic polymers sold under the names Neocryl XK-90®, Neocryl A-1070®, Neocryl A-1090®, Neocryl BT-62®, Neocryl A-1079® and Neocryl A-523® by the company Avecia-Neoresins, Dow Latex 432® by the company Dow Chemical, or polyurethanes such as the polyester-polyurethanes sold under the names “Avalure UR-405®”, “Avalure UR-410®”, “Avalure UR-425®” and “Sancure 2060®” by the company Goodrich, the polyether-polyurethanes sold under the names “Sancure 878®” and “Avalure UR-450®” by the company Goodrich and “Neorez R-970®” by the company ICI and the polyurethane-acrylics sold under the name Neorez R-989® by the company Avecia-Neoresins.

It is also possible to use “alkali-soluble” polymers, taking care to ensure that the pH of the composition is adjusted so as to keep these polymers in the form of particles in aqueous dispersion.

The composition according to the invention may comprise a film-forming auxiliary agent which promotes the formation of a film with the particles of the film-forming polymer. Such a film-forming agent may be chosen from any compounds known to those skilled in the art as being capable of fulfilling the desired function, and may be chosen from plasticizers and coalescers.

According to another embodiment of the invention, the film-forming polymer may be present in the form of surface-stabilized particles dispersed in a liquid fatty phase.

For example, the liquid fatty phase comprises a volatile liquid fatty phase, optionally mixed with a non-volatile liquid fatty phase.

The expression “volatile fatty phase” means any non-aqueous medium which is capable of evaporating from the skin in less than one hour. This volatile phase can comprise oils with a vapour pressure, at room temperature and atmospheric pressure, ranging from 10-3 to 300 mmHg (0.13 Pa to 40,000 Pa).

The liquid fatty phase in which the polymer is dispersed may comprise any physiologically acceptable and cosmetically acceptable oil, such as those chosen from oils of mineral, animal, plant or synthetic origin, carbon-based oils, hydrocarbon-based oils, fluoro oils and/or silicone oils, alone or as a mixture provided that they form a homogeneous and stable mixture and provided that they are compatible with the intended use.

The total liquid fatty phase of the composition may represent from 5% to 98% by weight relative to the total weight of the composition, such as from 20% to 85% by weight. The non-volatile part may represent from 0 to 80%, such as from 0.1% to 80%) of the total weight of the composition, and further such as from 1% to 50%.

As liquid fatty phase which may be used in the invention, mention may thus be made of fatty acid esters, higher fatty acids, higher fatty alcohols, polydimethylsiloxanes (PDMSs), which are optionally phenylated such as phenyltrimethicones, or which are optionally substituted with aliphatic and/or aromatic groups, which may be fluorinated, or are optionally substituted with functional groups such as hydroxyl, thiol and/or amine groups; polysiloxanes modified with fatty acids, with fatty alcohols or with polyoxyalkylenes, fluorosilicones and perfluoro oils.

For example, one or more oils that are volatile at room temperature may be used. After evaporating off these oils, a non-sticky, supple film-forming deposit is obtained. These volatile oils also make it easier to apply the composition to keratin fibres such as the eyelashes.

These volatile oils can be hydrocarbon-based oils or silicone oils optionally comprising alkyl or alkoxy groups at the end of the silicone chain or pendent on the chain.

As volatile silicone oils which can be used in the invention, mention may be made of linear or cyclic silicones comprising from 2 to 7 silicon atoms, these silicones optionally comprising alkyl or alkoxy groups comprising from 1 to 10 carbon atoms. Mention may be made, for example, of octamethylcyclotetrasiloxane, decamethylcyclopentasiloxane, hexadecamethylcyclohexasiloxane, heptamethylhexyltrisiloxane, and heptamethyloctyltrisiloxane.

Volatile hydrocarbon-based oils which may be mentioned are C8-C16 isoparaffins such as Isopars and Permetyls and isododecane, for instance.

These volatile oils can be present in the composition in a content ranging from 5 to 94.9% relative to the total weight of the composition, such as from 20 to 85%.

In one embodiment of the invention, the liquid fatty phase is chosen from the group comprising:

non-aqueous liquid compounds having a global solubility parameter according to the Hansen solubility space of less than 17 (MPa)1/2,

or monoalcohols having a global solubility parameter according to the Hansen solubility space of less than or equal to 20 (MPa)1/2,

or mixtures thereof.

The global solubility parameter δ global according to the Hansen solubility space is defined in the article “Solubility parameter values” by Eric A. Grulke in the book “Polymer Handbook” 3rd Edition, Chapter VII, pages 519-559, the disclosure of which is incorporated herein by reference, by the relationship:

δ=(dD2+dP2+dH2)1/2

in which

dD characterizes the London dispersion forces arising from the formation of dipoles induced during molecular impacts,

dP characterizes the Debye interaction forces between permanent dipoles,

dH characterizes the forces of specific interactions (such as hydrogen bonding, acid/base bonding, donor/acceptor bonding, etc.). The definition of solvents in the three-dimensional solubility space according to Hansen is described in the article, the disclosure of which is incorporated herein by reference, by C. M. Hansen: “The three-dimensional solubility parameters”, J. Paint Technol. 39, 105 (1967).

Oils which may be used in the liquid fatty phase are mentioned, for example, in patent application EP-A-749 747, the disclosure of which is incorporated herein by reference. Non-aqueous media which can also be used are those disclosed in document FR-A-2 710 646 from L.V.M.H., the disclosure of which is incorporated herein by reference.

The choice of the non-aqueous medium is made by a person skilled in the art on the basis of the nature of the monomers comprising the polymer and/or the nature of the stabilizer, as indicated below.

The polymer dispersion may be manufactured as disclosed in document EP-A-749 747, the disclosure of which is incorporated herein by reference. The polymerization may be carried out in dispersion, that is to say by precipitating the polymer as it is formed, with protection of the particles formed with a stabilizer.

The polymer particles in dispersion in the said fatty phase can have a size ranging from 5 nm to 600 nm, such as from 50 nm to 250 nm. One skilled in the art can use known methods to determine these sizes.

The polymer particles are surface-stabilized by means of a stabilizer which may be a block polymer, a grafted polymer and/or a random polymer, alone or as a mixture.

Among the grafted polymers which may be mentioned are silicone polymers grafted with a hydrocarbon-based chain and hydrocarbon-based polymers grafted with a silicone chain.

Copolymers that are also suitable are grafted copolymers having, for example, an insoluble skeleton of polyacrylic type with soluble grafts of poly(12-hydroxystearic acid) type.

Grafted block copolymers or block copolymers comprising at least one block of polyorganosiloxane type and at least one block of a free-radical polymer, for instance grafted copolymers of acrylic/silicone type which may be used when the non-aqueous medium is a silicone medium, for example.

The stabilizer may also be chosen from grafted block copolymers or block copolymers comprising at least one block of polyorganosiloxane type and at least one block of a polyether. The polyorganopolysiloxane block may be, for example, a polydimethylsiloxane or a poly(C2-C18)alkylmethylsiloxane; the polyether block may be a poly(C2-C18)alkylene, such as polyoxyethylene and/or polyoxypropylene. For example, dimethicone copolyols or (C2-C18)alkylmethicone copolyols may be used. It is possible, for example, to use the dimethicone copolyol sold under the name “Dow Corning 3225C” by the company Dow Corning or the laurylmethicone copolyol sold under the name “Dow Corning Q2-5200” by the company Dow Corning.

Grafted block copolymers or block copolymers which may be used include copolymers comprising at least one block resulting from the polymerization of at least one ethylenic monomer comprising one or more optionally conjugated ethylenic bonds, such as ethylene, butadiene or isoprene, and of at least one block of a styrene polymer. When the ethylenic monomer comprises several optionally conjugated ethylenic bonds, the residual ethylenic unsaturations after the polymerization are generally hydrogenated. Thus, in a known manner, the polymerization of isoprene leads, after hydrogenation, to the formation of an ethylene-propylene block, and the polymerization of butadiene leads, after hydrogenation, to the formation of an ethylene-butylene block. Among these block copolymers which may be mentioned are copolymers of “diblock” or “triblock” type such as polystyrene/polyisoprene or polystyrene/polybutadiene, such as those sold under the name “Luvitol HSB” by BASF, of the polystyrene/copoly(ethylene-propylene) type such as those sold under the name “Kraton” by Shell Chemical Co. or of the polystyrene/copoly(ethylene-butylene) type.

As grafted block copolymers or block copolymers comprising at least one block resulting from the polymerization of at least one ethylenic monomer, such as ethylene or isobutylene, and of at least one block of an acrylic polymer such as methyl methacrylate, mention may be made of poly(methyl methacrylate)/polyisobutylene diblock or triblock copolymers or grafted copolymers comprising a poly(methyl methacrylate) skeleton and polyisobutylene grafts.

As grafted block copolymers or block copolymers comprising at least one block resulting from the polymerization of at least one ethylenic monomer and of at least one block of a polyether such as a C2-C18 polyoxyalkylene, and further such as polyoxyethylene and/or polyoxypropylene, mention may be made of polyoxyethylene/polybutadiene or polyoxyethylene/polyisobutylene diblock or triblock copolymers.

It is also possible to use copolymers of C1-C4 alkyl (meth)acrylates and of C8-C30 alkyl (meth)acrylates. Mention may be made, for instance, of the stearyl methacrylate/methyl methacrylate copolymer.

In this case, a grafted polymer or a block polymer may be used as stabilizer, so as to have better interfacial activity. The reason for this is that the blocks or grafts that are insoluble in the synthesis solvent provide greater coverage at the surface of the particles.

When the liquid fatty phase comprises at least one silicone oil, the stabilizer can be chosen from grafted block copolymers and block copolymers comprising at least one block chosen from polyorganosiloxane types, free-radical polymers, polyethers, and polyesters, for instance, polyoxy(C2-C18)alkylene blocks and polyoxypropylenated and/or polyoxyethylenated blocks.

When the liquid fatty phase comprises no silicone oil, the stabilizer can be chosen, for instance, from the following:

(a) grafted block copolymers and block copolymers comprising at least one block of polyorganosiloxane type and at least one block chosen from free-radical polymers, polyethers, and polyesters,

(b) copolymers of C1-C4 alkyl acrylates and methacrylates and of C8-C30 alkyl acrylates and methacrylates,

(c) grafted block copolymers and block copolymers comprising at least one block resulting from the polymerization of at least one ethylenic monomer comprising conjugated ethylenic bonds,

and at least one block chosen from vinyl polymers, acrylic polymers, polyethers, and polyesters.

Diblock polymers can be used as stabilizers.

According to another embodiment of the invention, the second film-forming polymer may be present in dissolved form in a liquid fatty phase as defined above, which is also referred to as a liposoluble polymer.

Examples of liposoluble polymers which may be mentioned are polymers corresponding to formula (I) below:

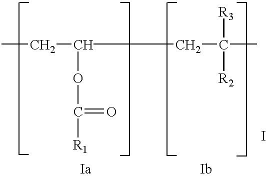

in which:

R1 is chosen from linear and branched saturated hydrocarbon-based chains comprising from 1 to 19 carbon atoms;

R2 is chosen from:

a) —O—CO—R4, R4 having the same meaning as R1 but is different from R1 in the same copolymer,

b) —CH2-R5, R5 is chosen from linear and branched saturated hydrocarbon-based chains comprising from 5 to 25 carbon atoms,

c) —O—R6, R6 is chosen from saturated hydrocarbon-based chains comprising from 2 to 18 carbon atoms, and

d) —CH2-O—CO—R7, R7 is chosen from linear and branched saturated hydrocarbon-based chains comprising from 1 to 20 carbon atoms,

R3 represents a hydrogen atom when R2 is chosen from radicals a), b), and c) or R3 represents a methyl radical when R2 is chosen from radicals d), the said copolymer needing to comprise at least 15% by weight of at least one monomeric residue chosen from units (Ia) and (Ib) in which the linear and branched saturated hydrocarbon-based chains comprise at least 7 carbon atoms.

The copolymers of formula (I) result from the copolymerization of at least one vinyl ester (corresponding to the unit Ia) and of at least one other monomer (corresponding to the unit Ib) which may be an α-olefin, an alkyl vinyl ether or an allylic or methallylic ester.

When, in the unit (Ib), R2 is chosen from the radicals —CH2-R5, —O—R6 or —CH2-O—CO—R7 as defined above, the copolymer of formula (I) may comprise from 50 mol % to 95 mol % of at least one unit (Ia) and from 5 mol % to 50 mol % of at least one unit (Ib).

The copolymers of formula (I) may also result from the copolymerization of at least one vinyl ester and of at least one other vinyl ester which is different from the first ester. In this case, these copolymers may comprise from 10 mol % to 90 mol % of at least one unit (Ia) and from 10 mol % to 90 mol % of at least one unit (Ib) in which R2 represents a radical —O—CO—R4.

Among the vinyl esters leading to the unit of formula (Ia), or to the unit of formula (Ib) in which R2 =—O—CO—R4, mention may be made of vinyl acetate, vinyl propionate, vinyl butanoate, vinyl octanoate, vinyl decanoate, vinyl laurate, vinyl stearate, vinyl isostearate, vinyl 2,2-dimethyloctanoate, and vinyl dimethylpropionate.

Among the α-olefins leading to the unit of formula (Ib) in which R2 =—CH2-R5, mention may be made of 1-octene, 1-dodecene, 1-octadecene and 1-eicosene, and mixtures of α-olefins comprising from 22 to 28 carbon atoms.

Among the alkyl vinyl ethers leading to the unit of formula (Ib) in which R2 =—O—R6, mention may be made of ethyl vinyl ether, n-butyl vinyl ether, isobutyl vinyl ether, decyl vinyl ether, dodecyl vinyl ether, cetyl vinyl ether, and octadecyl vinyl ether.

Among the allylic or methallylic esters leading to the unit of formula (Ib) in which R2 =—CH2—O—CO—R7, mention may be made of allyl and methallyl acetates, propionates, dimethylpropionates, butyrates, hexanoates, octanoates, decanoates, laurates, 2,2-dimethylpentanoates, stearates, and eicosanoates.

The copolymers of formula (I) may also be crosslinked using certain types of crosslinking agents which are intended to substantially increase their molecular weight.

This crosslinking is carried out during the copolymerization and the crosslinking agents may be either of the vinyl type or of the allylic or methallylic type. Among these crosslinking agents which may be mentioned are tetraallyloxyethane, divinylbenzene, divinyl octanedioate, divinyl dodecanedioate, and divinyl octadecanedioate, for example.

Among the various copolymers of formula (I) which may be used in the composition according to the invention, mention may be made of the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene, crosslinked with 0.2% divinylbenzene, and allyl propionate/allyl stearate, crosslinked with 0.2% divinylbenzene.

Liposoluble film-forming polymers which may also be mentioned include liposoluble homopolymers, such as those resulting from the homopolymerization of vinyl esters comprising from 9 to 22 carbon atoms or of alkyl acrylates or methacrylates, the alkyl radicals comprising from 10 to 20 carbon atoms.

Such liposoluble homopolymers may be chosen from polyvinyl stearate, polyvinyl stearate crosslinked with divinylbenzene, with diallyl ether or with diallyl phthalate, polystearyl (meth)acrylate, polyvinyl laurate or polylauryl (meth)acrylate, these poly(meth)acrylates possibly being crosslinked with the aid of ethylene glycol dimethacrylate or tetraethylene glycol dimethacrylate.

The liposoluble copolymers and homopolymers defined above are known and are disclosed for instance in patent application FR-A-2 232 303, the disclosure of which is incorporated herein by reference; they may have a weight-average molecular weight ranging from 2,000 to 500,000, such as from 4,000 to 200,000.

As liposoluble film-forming polymers which may be used in the invention, mention may also be made of polyalkylenes such as C2-C20 alkylene copolymers, other than the polyolefin wax defined in a), for instance polybutene, alkylcelluloses with a linear or branched, saturated or unsaturated C1 to C8 alkyl radical, for instance ethyl cellulose and propyl cellulose, vinylpyrrolidone (VP) copolymers and copolymers of vinylpyrrolidone and of C2 to C40 and of C3 to C20 alkene, for example. As examples of VP copolymers which may be used in the invention, mention may be made of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate copolymer.

The film-forming polymer may be present in a solids content ranging from 5% to 60% by weight relative to the total weight of the composition, such as from 10% to 45% by weight, and further such as from 15% to 35% by weight.

For example, the said organic compound and the hydrophobic film-forming polymer may be present in the composition in a hydrophobic film-forming polymer/organic compound weight ratio ranging from 20:1 to 0.1:1, such as from 10:1 to 0.5:1, and further such as from 8:1 to 1:1.

The composition may also comprise at least one dyestuff, for instance pulverulent compounds and/or liposoluble dyes, for example in a proportion of from 0.01% to 50% relative to the total weight of the composition. The pulverulent compounds may be chosen from the pigments and/or nacres usually used in cosmetic compositions. For example, the pulverulent compounds can represent from 0.1% to 25% of the total weight of the composition, such as from 1% to 20%.

The pigments may be white or coloured, and mineral and/or organic. Among the mineral pigments which may be mentioned are titanium dioxide, optionally surface-treated, zirconium oxide and cerium oxide, and also iron oxide, chromium oxide, manganese violet, ultramarine blue, chromium hydrate and ferric blue. Among the organic pigments which may be mentioned are carbon black, pigments of D & C type and lakes based on cochineal carmine or on barium, strontium, calcium or aluminium.

The nacreous pigments may be chosen from white nacreous pigments such as mica coated with titanium or with bismuth oxychloride, coloured nacreous pigments such as titanium mica with iron oxides, titanium mica with ferric blue or with chromium oxide, for instance, titanium mica with an organic pigment of the abovementioned type, and also nacreous pigments based on bismuth oxychloride.

The composition may also comprise fillers which may be chosen from those that are well known to those skilled in the art and which are commonly used in cosmetic compositions. The fillers may be mineral or organic, and lamellar or spherical. Mention may be made of talc, mica, silica, kaolin, Nylon powder (Orgasol from Atochem), poly-β-alanine powder, polyethylene powder, Teflon, lauroyllysine, starch, boron nitride, tetrafluoroethylene polymer powders, hollow microspheres such as Expancel (Nobel Industrie), Polytrap (Dow Corning), silicone resin microbeads (for example Tospearls from Toshiba), precipitated calcium carbonate, magnesium carbonate, magnesium hydrocarbonate, hydroxyapatite, hollow silica microspheres (Silica Beads from Maprecos), glass or ceramic microcapsules, metal soaps derived from organic carboxylic acids comprising from 8 to 22 carbon atoms, such as from 12 to 18 carbon atoms, for example zinc stearate, magnesium stearate, lithium stearate, zinc laurate or magnesium myristate.

The composition may also comprise any additive usually used in such compositions, such as thickeners, preserving agents, fragrances, sunscreens, free-radical scavengers, waxes, oils, moisturizers, vitamins, proteins, plasticizers, sequestrants, ceramides, acidifying or basifying agents, emollients.

Needless to say, a person skilled in the art will take care to select this or these optional additional compound(s), and/or the amount thereof, such that the advantageous properties of the composition according to the invention are not, or are not substantially, adversely affected by the addition envisaged.

The invention is illustrated in greater detail in the examples which follow.

### EXAMPLES 1 to 8

Mascaras having the composition below were prepared:

| | | |
|---|---|---|
| Polyurethane as an aqueous dispersion, | 18 | g A.M. |
| sold under the name Avalure UR 425 by | | |
| the company Goodrich, containing 49% by | | |
| weight of active materials | | |
| Hydroxyethylcellulose | 1.9 | g |
| Organic compound | 3 | g |
| Black iron oxide | 5 | g |
| Propylene glycol | 5 | g |
| Preserving agents qs | | |
| Water qs | 100 | g |

| | | | Organic | Inorganic |
|---|---|---|---|---|
| Example | Organic compound | IOB | part | part |
| 1 | Butyl stearate | 0.136 | 440 | 60 |
| 2 | Cetyl palmitate | 0.094 | 640 | 60 |
| 3 | Methyl palmitate | 0.176 | 340 | 60 |
| 4 | Methyl stearate | 0.158 | 380 | 60 |
| 5 | Stearyl alcohol | 0.278 | 360 | 100 |

For each composition, the resistance to cold water (20° C.) and to warm water (45° C. ) was measured in accordance with the following protocol:

A fringe of black Caucasian hair 2 cm wide and 1.5 cm long was bonded to a 2.5 cm×5 cm plate. The hair was made up with the test composition using a mascara brush by performing 2 times 10 applications with an interval of 2 minutes. The composition was left to dry for 2 hours at room temperature (25° C.).

A compress of cotton wool soaked with water (cold water or warm water) was then applied to the made-up hair for 10 seconds, after which the fringe of made-up hair was rubbed along its length with the soaked compress. This operation was carried out in this way in cycles of 10 consecutive rubbings.

The number of cycles after which the made-up fringe is completely freed of make-up was thus determined for each test composition.

The following results, expressed as the number of cycles of 10 rubbings, were obtained:

| Example | 1 | 2 | 3 | 4 | 5 |
|---|---|---|---|---|---|
| 20° C. | 5 | 9 | 5 | 6 | >10 |
| 40° C. | 2.5 | 4 | 2 | 2 | 3 |

It is found that, for each composition, the film is much less resistant in the presence of water at 40° C. (warm water) than in the presence of water at room temperature (cold water). The film is thus easier to remove with warm water and is more resistant to cold water.

### EXAMPLE 6

A mascara having the composition below was prepared:

| | | |
|---|---|---|
| Polyurethane as an aqueous dispersion, | 18 | g A.M. |
| sold under the name Avalure UR 425 by | | |
| the company Goodrich, at an active | | |
| material content of 49% by weight | | |
| Butyl stearate | 3 | g |
| Ethyl alcohol | 5 | g |
| Hydroxyethylcellulose | 1.9 | g |
| Propylene glycol | 5 | g |
| Pigments | 20 | g |
| Preserving agents qs | | |
| Water qs | 100 | g |

This mascara applies easily to the eyelashes and shows good resistance to cold water. It is easily removed with warm water (40° C.).

The measurements of the resistance to cold water and of the resistance to warm water of this mascara, carried out according to the protocol described in Examples 1 to 5, give the following results:

Resistance to cold water (20° C.): >10 cycles

Resistance to warm water (45° C.): 4 cycles

We claim:

1. A process of making a warm water removable film, in a composition comprising a physiologically acceptable medium, for application to a keratin material comprising:
forming said film by including at least one hydrophobic film-forming polymer and at least one nonionic organic compound in said composition, wherein said at least one nonionic organic compound has a melting point ranging from 20° C. to 70° C., and wherein said nonionic organic compound has an IOB (Inorganic/Organic Balance) value of less than or equal to 0.3.

2. The process according to claim 1, wherein said at least one nonionic organic compound has an IOB ranging from 0.05 to 0.3.

3. The process according to claim 1, wherein said at least one nonionic organic compound has an IOB ranging from 0.1 to 0.2.

4. The process according to claim 1, wherein said at least one nonionic organic compound has an organic value ranging from 330 to 700.

5. The process according to claim 4, wherein said at least one nonionic organic compound has an organic value ranging from 365 to 700.

6. The process according to claim 5, wherein said at least one nonionic organic compound has an organic value ranging from 365 to 500.

7. The process according to claim 1, wherein said at least one nonionic organic compound has an inorganic value ranging from 0 to 100.

8. The process according to claim 7, wherein said at least one nonionic organic compound has an inorganic value ranging from 50 to 80.

9. The process according to claim 1, wherein said at least one nonionic organic compound has a molecular weight ranging from 180 to 1,000.

10. The process according to claim 1, wherein said at least one nonionic organic compound is chosen from esters and mono-alcohols.

11. The process according to claim 10, wherein said at least one nonionic organic compound is chosen from esters comprising from 16 to 36 carbon atoms.

12. The process according to claim 1, wherein said at least one nonionic organic compound is chosen from butyl stearate, cetyl palmitate, methyl palmitate, and methyl stearate.

13. The process according to claim 10, wherein said at least one nonionic organic compound is chosen from monoalcohols comprising from 18 to 36 carbon atoms.

14. The process according to claim 13, wherein said at least one nonionic organic compound is stearyl alcohol.

15. The process according to claim 1, wherein said at least one nonionic organic compound is present in a content ranging from 0.1% to 20% by weight relative to the total weight of the composition.

16. The process according to claim 15, wherein said at least one nonionic organic compound is present in a content ranging from 0.5% to 15% by weight relative to the total weight of the composition.

17. The process according to claim 16, wherein said at least one nonionic organic compound is present in a content ranging from 1% to 10% by weight relative to the total weight of the composition.

18. The process according to claim 1, wherein said at least one hydrophobic film-forming polymer is chosen from free-radical polymers, polycondensates, and polymers of natural origin.

19. The process according to claim 1, wherein said at least one hydrophobic film-forming polymer is chosen from vinyl polymers, polyurethanes, polyesters, and cellulose polymers.

20. The process according to claim 1, wherein said at least one hydrophobic film-forming polymer is present in the form of particles dispersed in an aqueous medium.

21. The process according to claim 1, wherein said at least one hydrophobic film-forming polymer is chosen from polyurethanes in the form of particles in aqueous dispersion.

22. The process according to claim 1, wherein said at least one hydrophobic film-forming polymer is chosen from surface-stabilized particles dispersed in a liquid fatty phase.

23. The process according to claim 22, wherein said surface-stabilized particles are chosen from particles stabilized with at least one stabilizer chosen from block polymers, grafted polymers, and random polymers.

24. The process according to claim 23, wherein said at least one stabilizer is chosen from grafted block polymers and block polymers, comprising at least one block resulting from the polymerization of at least one ethylenic monomer comprising at least one optionally conjugated ethylenic bond and at least one block of a styrene polymer.

25. The process according to claim 1, wherein said at least one hydrophobic film-forming polymer is present in a solids content ranging from 5% to 60% by weight relative to the total weight of the composition.

26. The process according to claim 25, wherein said at least one hydrophobic film-forming polymer is present in a solids content ranging from 10% to 45% by weight relative to the total weight of the composition.

27. The process according to claim 1, wherein said at least one hydrophobic film-forming polymer and said at least one nonionic organic compound are present in the composition in a hydrophobic film-forming polymer/nonionic organic compound weight ratio ranging from 0.1:1 to 20:1.

28. The process according to claim 27, wherein said at least one hydrophobic film-forming polymer and said at least one nonionic organic compound are present in the composition in a hydrophobic film-forming polymer/nonionic organic compound weight ratio ranging from 0.5:1 to 10:1.

29. The process according to claim 28, wherein said at least one hydrophobic film-forming polymer and said at least one nonionic organic compound are present in the composition in a hydrophobic film-forming polymer/nonionic organic compound weight ratio ranging from 1:1 to 8:1.

30. A process of making a cold water resistant film, in a composition comprising a physiologically acceptable medium, for application to a keratin material comprising:
forming said film by including at least one hydrophobic film-forming polymer and at least one nonionic organic compound in said composition, wherein said at least one nonionic organic compound has a melting point ranging from 20° C. to 70° C., and wherein said nonionic organic compound has an IOB (Inorganic/Organic Balance) value of less than or equal to 0.3.

31. The process according to claim 30, wherein said cold water resistant film is additionally removable by warm water.

32. A composition for forming a film comprising, in a physiologically acceptable medium, at least one hydrophobic film-forming polymer and at least one nonionic organic compound, wherein said at least one nonionic organic compound has a melting point ranging from 20° C. to 70° C. and an IOB (Inorganic/Organic Balance) value of less than or equal to 0.3, and wherein said film is resistant to cold water.

33. The composition according to claim 32, wherein said at least one nonionic organic compound has an IOB ranging from 0.05 to 0.3.

34. The composition according to claim 33, wherein said at least one nonionic organic compound has an IOB ranging from 0.1 to 0.2.

35. The composition according to claim 32, wherein said at least one nonionic organic compound has an organic value ranging from 330 to 700.

36. The composition according to claim 35, wherein said at least one nonionic organic compound has an organic value ranging from 365 to 700.

37. The composition according to claim 36, wherein said at least one nonionic organic compound has an organic value ranging from 365 to 500.

38. The composition according to claim 32, wherein said at least one nonionic organic compound has an inorganic value ranging from 0 to 100.

39. The composition according to claim 38, wherein said at least one nonionic organic compound has an inorganic value ranging from 50 to 80.

40. The composition according to claim 32, wherein said at least one nonionic organic compound has a molecular weight ranging from 180 to 1,000.

41. The composition according to claim 32, wherein said at least one nonionic organic compound is chosen from esters and monoalcohols.

42. The composition according to claim 41, wherein said at least one nonionic organic compound is chosen from esters comprising from 16 to 36 carbon atoms.

43. The composition according to claim 32, wherein said at least one nonionic organic compound is chosen from butyl stearate, cetyl palmitate, methyl palmitate, and methyl stearate.

44. The composition according to claim 41, wherein said at least one nonionic organic compound is chosen from monoalcohols comprising from 18 to 36 carbon atoms.

45. The composition according to claim 44, wherein said at least one nonionic organic compound is stearyl alcohol.

46. The composition according to claim 32, wherein said at least one nonionic organic compound is present in a content ranging from 0.1% to 20% by weight relative to the total weight of the composition.

47. The composition according to claim 46, wherein said at least one nonionic organic compound is present in a content ranging from 0.5% to 15% by weight relative to the total weight of the composition.

48. The composition according to claim 47, wherein said at least one nonionic organic compound is present in a content ranging from 1% to 10% by weight relative to the total weight of the composition.

49. The composition according to claim 32, wherein said at least one hydrophobic film-forming polymer is chosen from free-radical polymers, polycondensates, and polymers of natural origin.

50. The composition according to claim 32, wherein said at least one hydrophobic film-forming polymer is chosen from vinyl polymers, polyurethanes, polyesters, and cellulose polymers.

51. The composition according to claim 32, wherein said at least one hydrophobic film-forming polymer is present in the form of particles dispersed in an aqueous medium.

52. The composition according to claim 32, wherein said at least one hydrophobic film-forming polymer is chosen from polyurethanes in the form of particles in aqueous dispersion.

53. The composition according to claim 32, wherein said at least one hydrophobic film-forming polymer is chosen from surface-stabilized particles dispersed in a liquid fatty phase.

54. The composition according to claim 53, wherein said surface-stabilized particles are chosen from particles stabilized with at least one stabilizer chosen from block polymers, grafted polymers, and random polymers.

55. The composition according to claim 54, wherein said at least one stabilizer is chosen from grafted block polymers and block polymers, comprising at least one block resulting from the polymerization of at least one ethylenic monomer comprising at least one optionally conjugated ethylenic bonds and at least one block of a styrene polymer.

56. The composition according to claim 32, wherein said at least one hydrophobic film-forming polymer is present in a solids content ranging from 5% to 60% by weight relative to the total weight of the composition.

57. The composition according to claim 56, wherein said at least one hydrophobic film-forming polymer is present in a solids content ranging from 10% to 45% by weight relative to the total weight of the composition.

58. The composition according to claim 32, wherein said at least one hydrophobic film-forming polymer and said at least one nonionic organic compound are present in said composition in a hydrophobic film-forming polymer/nonionic organic compound weight ratio ranging from 0.1:1 to 20:1.

59. The composition according to claim 58, wherein said at least one hydrophobic film-forming polymer and said at least one nonionic organic compound are present in said composition in a hydrophobic film-forming polymer/nonionic organic compound weight ratio ranging from 0.5:1 to 10:1.

60. The composition according to claim 59, wherein said at least one hydrophobic film-forming polymer and said at least one nonionic organic compound are present in said composition in a hydrophobic film-forming polymer/nonionic organic compound weight ratio ranging from 1:1 to 8:1.

61. The composition according to claim 32, further comprising at least one additive chosen from thickeners, dyestuffs, preserving agents, fragrances, sunscreens, free-radical scavengers, waxes, oils, moisturizers, vitamins, proteins, plasticizers, sequestrants, ceramides, acidifying and basifying agents, and emollients.

62. The composition according to claim 32, wherein said film is additionally removable by warm water.

63. A composition for forming a film comprising, in a physiologically acceptable medium, at least one hydrophobic film-forming polymer and at least one nonionic organic compound, wherein said at least one nonionic organic compound has a melting point ranging from 20° C. to 70° C. and an IOB (Inorganic/Organic Balance) value of less than or equal to 0.3, and wherein said film is removable by warm water.

64. A mascara, an eyeliner, a product for the lips, a blusher, an eyeshadow, a foundation, a make-up product for the body, a concealer product, a product for the nails, an antisun composition, a skin colouring composition, or a skincare product for forming a film comprising, in a physiologically acceptable medium, at least one hydrophobic film-forming polymer and at least one nonionic organic compound, wherein said at least one nonionic organic compound has a melting point ranging from 20° C. to 70° C. and an IOB (Inorganic/Organic Balance) value of less than or equal to 0.3, and wherein said film is resistant to cold water.

65. The mascara, eyeliner, product for the lips, blusher, eyeshadow, foundation, make-up product for the body, concealer product, product for the nails, antisun composition, skin colouring composition, or skincare product for forming a film according to claim 64, wherein said film is additionally removable by warm water.

66. A mascara for forming a film comprising, in a physiologically acceptable medium, at least one hydrophobic film-forming polymer and at least one nonionic organic compound, wherein said at least one nonionic organic compound has a melting point ranging from 20° C. to 70° C. and an IOB (Inorganic/Organic Balance) value of less than or equal to 0.3, and wherein said film is resistant to cold water.

67. The mascara according to claim 66, wherein said film is additionally removable by warm water.

68. A cosmetic care or make-up process for a keratin material, comprising: applying to said keratin material a film of composition comprising, in a physiologically acceptable medium, at least one hydrophobic film-forming polymer and at least one nonionic organic compound, wherein said at least one nonionic organic compound has a melting point ranging from 20° C. to 70° C. and an IOB (Inorganic/Organic Balance) value of less than or equal to 0.3, and wherein said film is resistant to cold water.

69. The cosmetic care or make-up process according to claim 68, wherein said film is additionally removable by warm water.

70. A method of forming a film on a keratin material comprising applying to said keratin material at least one hydrophobic film-forming polymer and at least one nonionic organic compound, wherein said at least one nonionic organic compound has a melting point ranging from 20° C. to 70° C., and wherein said nonionic organic compound has an IOB (Inorganic/Organic Balance) value of less than or equal to 0.3 to form said film, wherein said film is resistant to cold water and/or removable with warm water.

71. A cosmetic process for removing make-up from a keratin material comprising at least one step of rinsing, with warm water, from said keratin material a make-up comprising at least one hydrophobic film-forming polymer and at least one nonionic organic compound, wherein said at least one nonionic organic compound has a melting point ranging from 20° C. to 70° C., and wherein said nonionic organic compound has an IOB (Inorganic/Organic Balance) value of less than or equal to 0.3.

72. The process according to claim 71, wherein said warm water contains no detergent.
